(19) United States
(12) Patent Application Publication
(10) Pub. No.: US 2024/0261144 A1
(43) Pub. Date: Aug. 8, 2024

(54) BIOLOGICAL FLUID DRAINAGE DEVICES AND METHODS

(71) Applicant: W. L. Gore & Associates, Inc., Newark, DE (US)

(72) Inventors: Jeffrey C. Towler, Wilmington, DE (US); Jesse R. McAlister, Newark, DE (US); Kevin M. Savory, West Grove, PA (US)

(21) Appl. No.: 18/436,370

(22) Filed: Feb. 8, 2024

Related U.S. Application Data
(60) Provisional application No. 63/444,089, filed on Feb. 8, 2023.

(51) Int. Cl. A61F 9/007; A61L 31/04

(52) U.S. Cl. CPC A61F 9/00781; A61L 31/04; A61F 2210/0071; A61F 2220/005; A61F 2250/0024; A61F 2250/0029; A61F 2250/0036; A61F 2250/0051

(57) ABSTRACT

A drainage device for draining a biological fluid from an eye to a tissue external to the eye is implantable at least in part within a tissue of the eye. The drainage device includes a body portion which includes a membrane having a first surface with a first porosity and a second surface with a second porosity greater than the first porosity, and a plate having a porosity less than the first porosity. The membrane is more flexible than the plate and has a first thickness from 25 μm to 125 μm. The plate has a second thickness from 0.8 mm to 2.0 mm. The membrane and plate define a reservoir. A conduit fluidly coupled with the reservoir and insertable into the eye facilitates drainage of fluid into the conduit. The second surface of the membrane includes ingrowth surface region(s) for facilitating ingrowth of tissue external to the eye.

### CROSS-REFERENCE TO RELATED APPLICATION

This application claims the benefit of U.S. Provisional App. No. 63/444,089, filed Feb. 8, 2023, the disclosure of which is incorporated herein by reference in its entirety for all purposes.

### FIELD

The present disclosure generally relates to apparatuses and methods for draining biological fluid and diverting the biological fluid. More specifically, the disclosure relates to apparatuses and methods for draining aqueous humor from an anterior chamber (AC) of a patient's eye such that it may be reabsorbed by the body.

### BACKGROUND

Various medical interventions involve evacuating excess biological fluid from one portion of the body and redirecting it to another location of the body where it may be reabsorbed. In certain instances, this evacuation is achieved via minimally invasive procedures such as endoscopic third ventriculostomy (ETV) and choroid plexus cauterization procedure (CPC). In other instances, this evacuation is performed post-operatively via implantable medical devices, such as a shunt. Proven useful in various medical procedures, shunts of different forms have been employed as treatment for numerous diseases, such as hydrocephalus and glaucoma.

Without treatment, excessive biological fluid may lead to unhealthy pressure buildups. For instance, glaucoma is a progressive eye disease characterized by elevated intraocular pressure. Aqueous humor is a fluid that fills an anterior chamber (AC) of the eye and contributes to intraocular pressure or intraocular fluid pressure. This increase in intraocular pressure is usually caused by an insufficient amount of aqueous humor absorbed by the body. In some cases, the aqueous humor is not absorbed quickly enough or even not absorbed at all, while in other cases, the aqueous humor is additionally or alternatively produced too quickly. Elevated intraocular pressure is associated with gradual and sometimes permanent loss of vision in the affected eye.

### SUMMARY

Disclosed herein are devices for draining a biological fluid from an eye to a tissue surrounding the eye, as well as methods for forming such devices and for treating a glaucoma using such devices.

According to one example (“Example 1”), a drainage device for draining a fluid from an eye to a tissue external to the eye is disclosed, such that the drainage device is implantable at least in part within a tissue of the eye. The device includes a body portion which includes: a membrane having a first surface with a first porosity and a second surface with a second porosity that is greater than the first porosity, the second surface opposing the first surface, and a plate having a porosity less than the first porosity. The membrane is more flexible than the plate, and the membrane has a first thickness from about 25 μm to about 125 μm, and the plate has a second thickness from about 0.8 mm to about 2.0 mm. The membrane and the plate define a reservoir of the body portion. The device also includes a conduit having an end portion fluidly coupled with the reservoir and insertable into the eye to facilitate a drainage of the fluid into the conduit. The second surface of the membrane with the second porosity includes at least one ingrowth surface region for facilitating ingrowth of tissue external to the eye.

According to another example (“Example 2”) further to Example 1, the plate is nonporous.

According to another example (“Example 3”) further to Example 1 or 2, the plate includes one or more of: silicone or polypropylene.

According to another example (“Example 4”) further to any preceding Example, the membrane includes one or more of: expanded polytetrafluoroethylene (ePTFE), expanded polyethylene (ePE), silicone, polysulfone, polyvinylidene fluorine (PVDF), polyhexafluoropropylene (PHFP), perfluoroalkoxy polymer (PFA), polyolefin, fluorinated ethylene propylene (FEP), or acrylic copolymer.

According to another example (“Example 5”) further to any preceding Example, the device further includes an adhesive disposed between the membrane and the plate.

According to another example (“Example 6”) further to Example 5, the adhesive is a thermoplastic.

According to one example (“Example 7”), a drainage device for draining a fluid from an eye to a tissue external to the eye is disclosed. The drainage device being implantable at least in part within a tissue of the eye and includes a body portion which includes: a membrane having a porosity such that the membrane is configured to allow the fluid from the eye to pass therethrough and inhibit ingrowth of the tissue external to the eye, and a plate having a porosity less than the porosity of the membrane. The membrane is more flexible than the plate, and the membrane has a first thickness from about 25 μm to about 125 μm, and the plate has a second thickness from about 0.8 mm to about 2.0 mm. The membrane and the plate define a reservoir of the body portion. The device further includes a conduit having an end portion fluidly coupled with the reservoir and insertable into the eye to facilitate a drainage of the fluid into the conduit.

According to another example (“Example 8”) further to Example 7, the plate is nonporous.

According to another example (“Example 9”) further to Example 7 or 8, the plate includes one or more of: silicone or polypropylene.

According to another example (“Example 10”) further to any one of Examples 7-9, the membrane includes one or more of: expanded polytetrafluoroethylene (ePTFE), expanded polyethylene (ePE), silicone, polysulfone, polyvinylidene fluorine (PVDF), polyhexafluoropropylene (PHFP), perfluoroalkoxy polymer (PFA), polyolefin, fluorinated ethylene propylene (FEP), or acrylic copolymer.

According to another example (“Example 11”) further to any one of Examples 7-10, the device further includes an adhesive disposed between the membrane and the plate.

According to another example (“Example 12”) further to Example 11, the adhesive is a thermoplastic.

According to one example (“Example 13”), a drainage device for draining a fluid from an eye to a tissue external to the eye is disclosed, such that the drainage device is implantable at least in part within a tissue of the eye and includes a body portion including a membrane having a first surface with a first porosity and a second surface with a second porosity that is greater than the first porosity, the second surface opposing the first surface, and a plate having a porosity less than the first porosity. The membrane has a first thickness from about 25 μm to about 125 μm, and the plate has a second thickness from about 0.05 μm to about 10 μm, and the membrane and the plate define a reservoir of the body portion. The device further includes a conduit at least partially defined by the membrane and the plate, the conduit fluidly coupled with the reservoir and insertable into the eye to facilitate a drainage of the fluid into the conduit. The second surface of the first body component with the second porosity includes at least one ingrowth surface region for facilitating ingrowth of tissue external to the eye.

According to another example (“Example 14”) further to Example 13, the plate is nonporous.

According to another example (“Example 15”) further to Example 13 or 14, the plate includes one or more of: silicone or polypropylene.

According to another example (“Example 16”) further to any one of Examples 13-15, the membrane includes one or more of: expanded polytetrafluoroethylene (ePTFE), expanded polyethylene (ePE), silicone, polysulfone, polyvinylidene fluorine (PVDF), polyhexafluoropropylene (PHFP), perfluoroalkoxy polymer (PFA), polyolefin, fluorinated ethylene propylene (FEP), or acrylic copolymer.

According to another example (“Example 17”) further to any one of Examples 13-16, the device further includes an adhesive disposed between the membrane and the plate.

According to another example (“Example 18”) further to Example 17, the adhesive is a thermoplastic.

According to one example (“Example 19”), a drainage device for draining a fluid from an eye to a tissue external to the eye is disclosed, such that the drainage device is implantable at least in part within a tissue of the eye and includes a collapsible body portion including a first body component having a first surface with a first porosity and a second surface with a second porosity that is greater than the first porosity, the second surface opposing the first surface, and a second body component having a third porosity that is less than the first porosity. The first and second body components define a reservoir of the collapsible body portion. The device further includes a conduit having an end portion fluidly coupled with the reservoir and insertable into the eye to facilitate a drainage of the fluid into the conduit. The second surface of the first body component with the second porosity includes at least one ingrowth surface region for facilitating ingrowth of tissue external to the eye.

According to another example (“Example 20”) further to Example 19, the second body component is nonporous.

According to another example (“Example 21”) further to Example 19 or 20, the second body component includes one or more of: silicone or polypropylene.

According to another example (“Example 22”) further to any one of Examples 19-21, the second body component has a greater stiffness than first body component.

According to another example (“Example 23”) further to any one of Examples 19-22, the first body component includes one or more of: expanded polytetrafluoroethylene (ePTFE), expanded polyethylene (ePE), silicone, polysulfone, polyvinylidene fluorine (PVDF), polyhexafluoropropylene (PHFP), perfluoroalkoxy polymer (PFA), polyolefin, fluorinated ethylene propylene (FEP), or acrylic copolymer.

According to another example (“Example 24”) further to any one of Examples 19-23, the device further includes an adhesive disposed between first and second body components.

According to another example (“Example 25”) further to Example 24, the adhesive is a thermoplastic.

According to one example (“Example 26”), a drainage device for draining a fluid from an eye to a tissue external to the eye is disclosed, such that the drainage device is implantable at least in part within a tissue of the eye and includes a body portion including a first body component having a first surface with a first porosity and a second surface with a second porosity that is greater than the first porosity, the second surface opposing the first surface, and a second body component having a third porosity that is less than the first porosity and defining a reservoir of the body portion. The first surface of the first body component is attached to an outer surface of the second body component. The device further includes a conduit having an end portion fluidly coupled with the reservoir and insertable into the eye to facilitate a drainage of the fluid into the conduit. The second surface of the first body component with the second porosity includes at least one ingrowth surface region for facilitating ingrowth of tissue external to the eye, and the first surface of the first body component with the first porosity is configured to inhibit the ingrowth of tissue external to the eye.

According to another example (“Example 27”) further to Example 26, the second body component includes a plurality of subcomponents attached together.

According to another example (“Example 28”) further to Example 26 or 27, the device further includes an open-ended valve enclosed in the body portion. The valve is fluidly coupled with the conduit and partially defines the reservoir.

According to another example (“Example 29”) further to any one of Examples 26-28, the first surface with the first porosity is configured to inhibit the ingrowth of the tissue external to the eye.

According to another example (“Example 30”) further to any one of Examples 26-29, the second surface further includes at least one low-porosity region for inhibiting the ingrowth of the tissue external to the eye.

According to another example (“Example 31”) further to any one of Examples 26-30, the first body component includes a plurality of internal regions with porosities that are greater than the first porosity and less than the second porosity.

According to another example (“Example 32”) further to any one of Examples 26-31, the conduit is attached to a periphery of the body portion.

According to another example (“Example 33”) further to any one of Examples 26-31, the conduit is attached to the body portion across a portion of a cross-sectional length of the body portion.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIGS. 1A and 1B are schematic drawings of a cross-sectional view of a drainage device according to embodiments disclosed herein;

FIG. 1C is an SEM image of a portion of a cross-sectional view of the drainage device according to embodiments disclosed herein (the image is to the scale shown in the image);

FIG. 1D is a schematic drawing of a cross-sectional view of an eye when the drainage device is at least partially implanted according to embodiments disclosed herein;

FIG. 1E is a microscopic image of a tissue slide stained with a histological stain agent showing a cross-section of the drainage device according to embodiments disclosed herein (the image is to the scale shown in the image);

FIGS. 2A and 2B are schematic drawings of a bottom view and a top view, respectively, of drainage devices according to embodiments disclosed herein;

FIGS. 2C and 2D are schematic drawings of a cross-sectional view of drainage devices according to embodiments disclosed herein, as cut across line C/D-C/D of FIG. 2B;

FIGS. 3A and 3B are schematic drawings of a bottom view and a top view, respectively, of drainage devices according to embodiments disclosed herein;

FIGS. 3C and 3D are schematic drawings of a cross-sectional view of drainage devices according to embodiments disclosed herein, as cut across line C/D-C/D of FIG. 3B;

FIGS. 4A and 4B are schematic drawings of a bottom view and a top view, respectively, of drainage devices according to embodiments disclosed herein;

FIG. 4C is a schematic drawing of a cross-sectional view of a drainage device according to embodiments disclosed herein, as cut across line C-C of FIG. 4B;

FIG. 5A is a top view of a drainage device according to embodiments disclosed herein;

FIG. 5B is a cross-sectional view of the drainage device of FIG. 5A, as cut across line 5B-5B of FIG. 5A;

FIG. 5C is an enlarged view of a portion of the cross-sectional view of the drainage device of FIG. 5B;

FIG. 6A is a top view of a drainage device according to embodiments disclosed herein;

FIG. 6B is a cross-sectional view of the drainage device of FIG. 6A, as cut across line 6B-6B of FIG. 6A;

FIG. 6C is an enlarged view of a portion of the cross-sectional view of the drainage device of FIG. 6B;

FIG. 7A (prior art) is an illustration of a top view of a prior-art glaucoma drainage device;

FIG. 7B (prior art) is an illustration of a side view of the prior-art glaucoma drainage device of FIG. 7A;

FIG. 7C (prior art) is an illustration of an angled view of the prior-art glaucoma drainage device of FIG. 7A;

FIGS. 8A through 8C (prior art) are illustrations of a top view of a plurality of prior-art glaucoma devices having a solid and rigid plate;

FIG. 9A (prior art) is an illustration of a top view of an Ahmed® glaucoma valve modified with a polyethylene shell and implementing solid plates as known in the art;

FIG. 9B (prior art) is an illustration of a side view of the prior-art glaucoma valve of FIG. 9A when a portion of the solid plates is removed to show the reservoir located therein;

FIG. 9C (prior art) is an SEM image of a portion of a surface of the solid plate used in the prior-art glaucoma valve of FIG. 9A (the image is to the scale shown in the image).

It should be understood that the drawings and replicas of the photographs are not necessarily to scale, unless otherwise indicated. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The term “fibril” as used herein describes an elongated piece of material such as a polymer, where the length and width are substantially different from each other. For example, a fibril may resemble a piece of string or fiber, where the width (or thickness) is much shorter or smaller than the length.

The term “node” as used herein describes a connection point of at least two fibrils, where the connection may be defined as a location where the two fibrils come into contact with each other, permanently or temporarily. In some examples, a node may also be used to describe a larger volume of polymer than a fibril and where a fibril originates or terminates with no clear continuation of the same fibril through the node. In some examples, a node has a greater width but a smaller length than the fibril.

As used herein, “nodes” and “fibrils” may be used to describe objects that are usually, but not necessarily, connected or interconnected, and have a microscopic size, for example. A “microscopic” object may be defined as an object with at least one dimension (width, length, or height) that is substantially small such that the object or the detail of the object is not visible to the naked eye or difficult, if not impossible, to observe without the aid of a microscope (including but not limited to a scanning electron microscope or SEM, for example) or any suitable type of magnification device.

### Description of Various Embodiments

Various embodiments as disclosed herein address a drainage device which can be implanted in the eye to drain such fluid from the AC of an eye and accommodate for means of avoiding or preventing hypotony. The embodiments address various problems and issues faced by the prior-art drainage devices as currently used in the art, as explained further below.

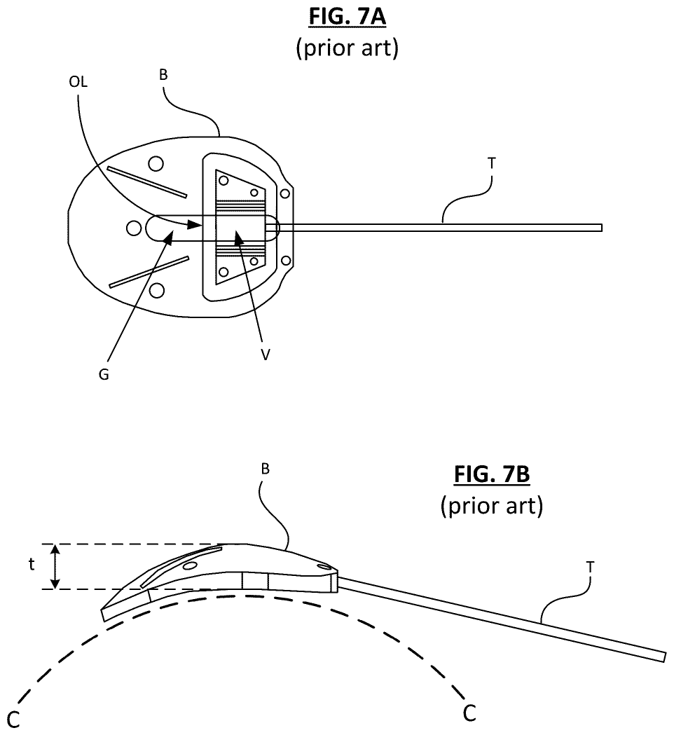

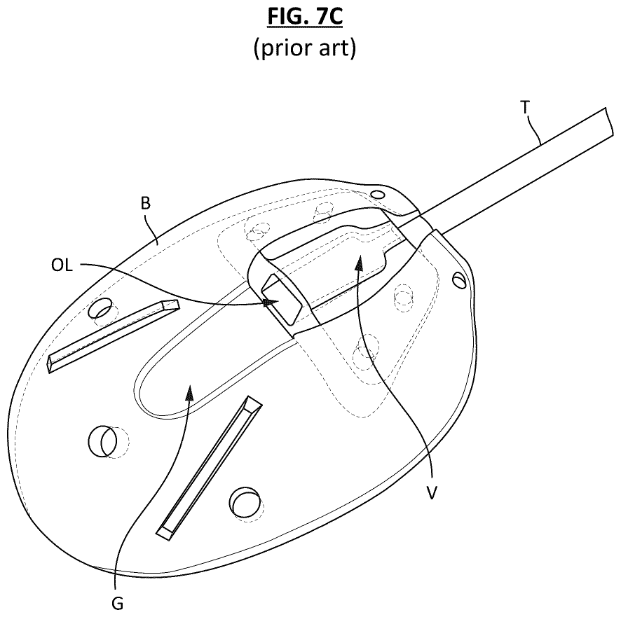

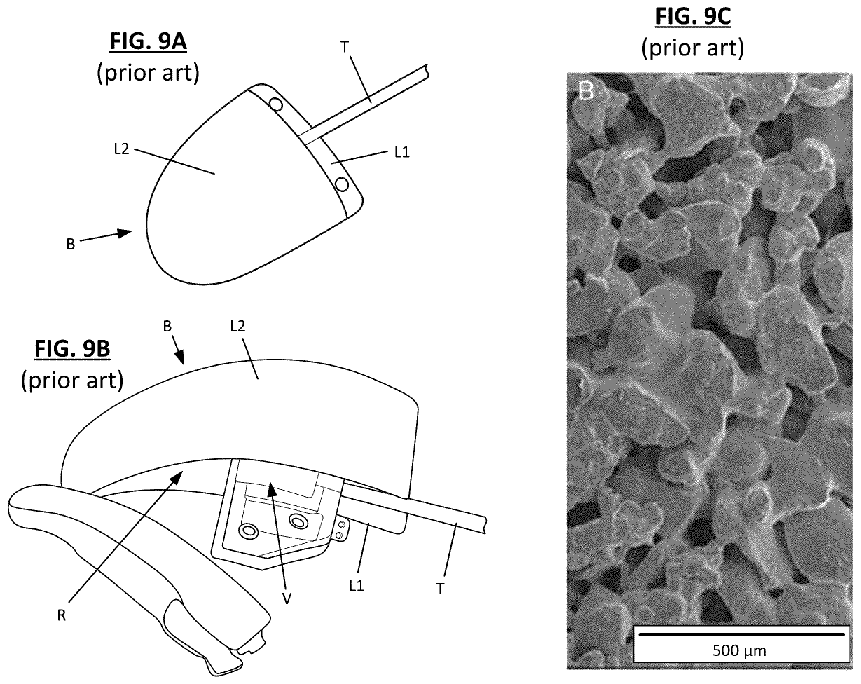

Some prior-art drainage devices may implement solid shells or plates, such as those made from silicone as shown in FIGS. 7A-C and 8A-C, or porous shells such as those made from polyethylene as shown in FIGS. 9A-C, to receive fluid from the AC of the eye and store it inside an internal chamber formed between the plates.

FIGS. 7A-C illustrate a glaucoma drainage device as known in the art. The device includes a plate body “B” which defines a surface over which the drained fluid (aqueous humor) is directed to flow, and a drainage tube “T” which directs the fluid (aqueous humor) to flow over the surface of the plate body. The implanted plate body B (of FIGS. 7A-C and 8A-C) defines a surface area for the fluid to be absorbed by the surrounding tissue, more specifically by the conjunctival tissue on the superior side of the plate body. In the art, this surface area is referred to as a “bleb”, and the absorption of redirected fluid into the surrounding is also referred to as filtering through the tissue, hence these devices are sometimes referred to as “filtration” devices. The plate body B has a maximum thickness “t1” which in the example shown is 2.1 mm, and is made of medical-grade silicone which lacks flexibility to conform to the curvature of the eye when implanted. As such, the plate body B has a preformed curvature (defined by the broken line C-C in FIG. 7B) to approximate the curvature of the surface of the eye. The curvature C-C is fixed and is the same for all glaucoma drainage devices, and thus fails to accommodate the unique curvature of each patient's eye. The plate body B also includes a valve “V” embedded therein, with an outlet “OL” which directs the fluid from the tube T toward a gutter “G” located on an outer surface of the plate body B as illustrated in FIG. 7C.

In the prior-art device of FIGS. 7A-C, which is an Ahmed® Glaucoma Valve model FP7 (New World Medical, Inc.; Rancho Cucamonga, CA), the plate body B has a structure that is stiff to allow the user to hold any part of the plate body B using tools such medical tweezers, hemostats, or any other suitable medical tool, and the user can push the plate body B into the tissue pocket in the eye which is made by the incision, while holding the plate body B with the medical tool. Likewise, the prior-art device of FIGS. 7A-C relies upon a slippery or low-friction surface of the device to facilitate insertion of the device via pushing into the tissue pocket in the eye by the surgeon or practitioner, and thus, any device with a higher surface friction would experience resistance against the pushing motion which would likely lead to deformation of the device during implantation, which would collapse (e.g., flex, fold, buckle, deform, or crumple) the device.

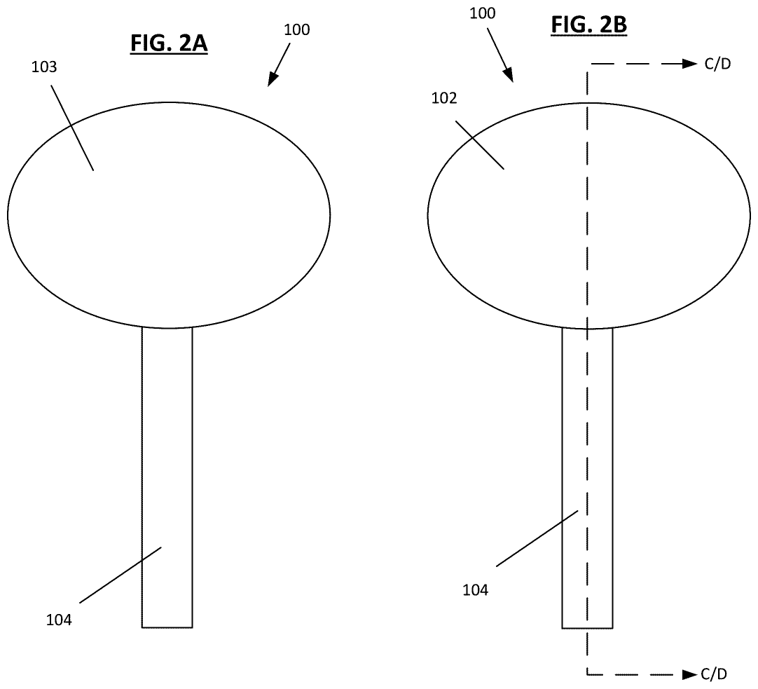

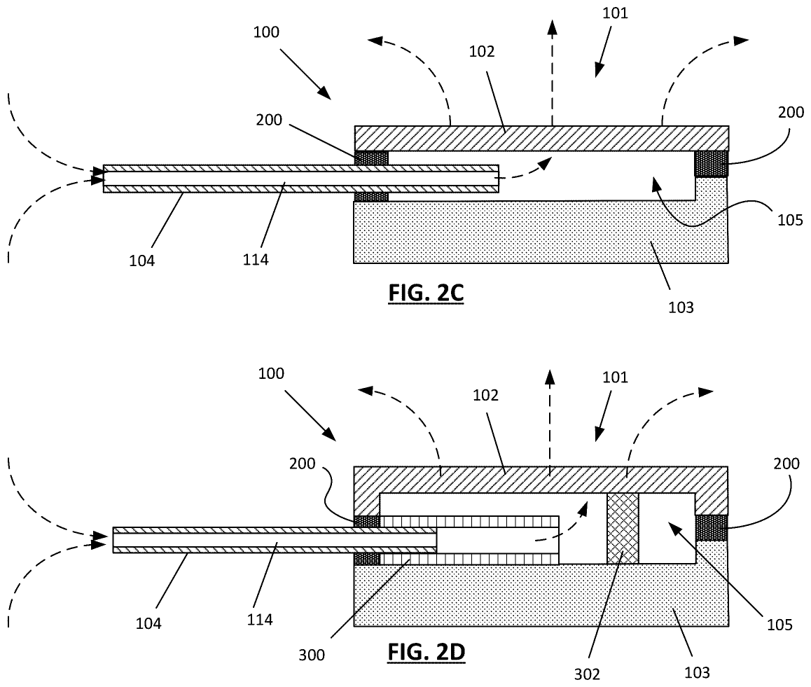

In the aforementioned Ahmed® model, the plate body B is made of medical grade silicone, and the casing of the valve V is made of medical grade polypropylene. The solid silicone plate body B does not integrate with the surrounding tissue when implanted, therefore the lack of integration causes a poor tissue response to alleviate the stress caused by the pressure exerted by the plate body B, leading to fibrosis that will plague the plate body B, in turn causing high complication rates over subsequent years after the device is implanted as further explained in the article: Christakis P G et al. “The Ahmed Versus Baerveldt Study: Five-Year Treatment Outcomes.” Ophthalmology. 2016 October; 123(10):2093-102. DOI: 10.1016/j.ophtha.2016.06.035. Epub 2016 Aug. 17. PMID: 27544023. For example, FIG. 2 of the article illustrates the cumulative failure rate of the Ahmed® model and the Baerveldt® Glaucoma Implant device model 101-350. As such, in contrast to the prior-art device, integration with the surrounding tissue helps reduce fibrosis and other problems after implanting a drainage device.

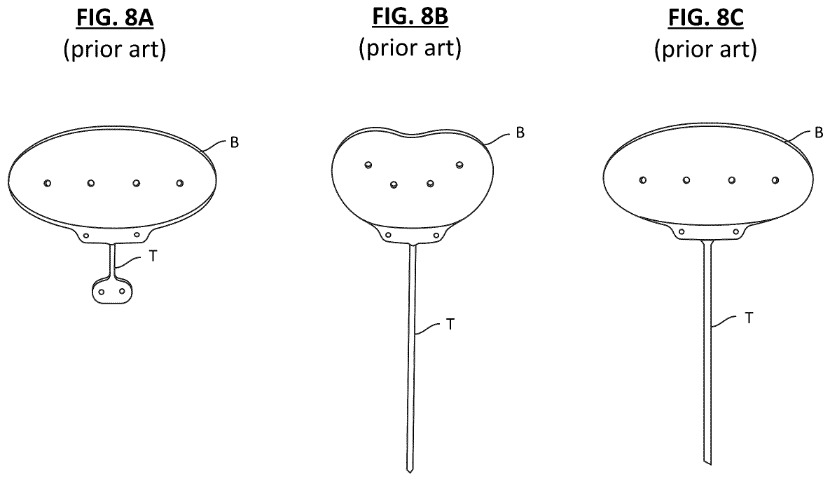

Other prior-art devices are shown in FIGS. 8A-C, each of which includes a plate body B and a tube T extending therefrom. The devices shown are Baerveldt® Glaucoma Implant devices (Johnson & Johnson Surgical Vision, Inc.; Irvine, CA), for example model BG 102-350 (FIG. 8A), BG 103-250 (FIG. 8B), and BG 101-350 (FIG. 8C). The plate body B is shaped with a predetermined curvature to generally accommodate the curvature of the eye into which the device is to be implanted, although the curvature is not customized to the specific eye of the patient. The plate bodies B are made of materials such as silicone plates impregnated with barium sulphate, for example. As with the Ahmed® devices of FIGS. 7A-C, the solid silicone plate body of the Baerveldt® devices also fails to provide means of integration of the plate body B with the surrounding tissues, causing similar problems and complications. The aforementioned article “The Ahmed Versus Baerveldt Study” also explains the high failure rate of the Baerveldt® devices such as the model BG 101-350. Additional devices which may fall under similar category, and therefore include similar problems as explained above, include: Ahmed ClearPath® (New World Medical, Inc.; Rancho Cucamonga, CA), Molteno3® (Nova Eye Medical Ltd.; Fremont, CA), PAUL® Glaucoma Implant (Advanced Ophthalmic Innovations Pte Ltd.; Singapore), eyePlate® (Rheon Medical SA; Switzerland), Keiki Mehta “BP Valve” glaucoma shunt (G. Surgiwear Ltd.; India), and Aurolab Aqueous Drainage Implants (“AADI”, Aurorab; India).

Another prior-art device shown in FIGS. 9A and 9B is an Ahmed® Glaucoma Valve model M4 (New World Medical, Inc.; Rancho Cucamonga, CA) with a polyethylene shell to reduce the fibrotic reaction around the drainage plate compared with the S2 and FP7 models in patients with glaucoma, as previously disclosed in Kim J, Allingham RR, Hall J, et al. “Clinical experience with a novel glaucoma drainage implant.” Journal of Glaucoma. 2014 February; 23(2):e91-7. DOI: 10.1097/ijg.0b013e3182955d73. PMID: 23689073. This prior-art device includes a body shell B and a tube T installed between two layers (L1 and L2) of solid material such as porous polyethylene shells (e.g., Medpor) that generally follow the curvature of the eye, although not customized to the specific eye of the patient, and between the layers L1 and L2 is defined a reservoir “R” into which the tube T directs the fluid from the eye. As shown in FIG. 9C, which is scaled such that the black bar on the bottom of the figure represents 500 μm, the surface of the layers L1 and L2 of the prior-art device includes pores to improve tissue integration, such that the adjacent tissue can be integrated into the polyethylene shell surrounding the drainage device, as well as to ensure that fluid stored inside the reservoir R can be released back to the surrounding environment in order to prevent the shell from applying undue stress on the surrounding tissue. The implanted body shell B (of FIGS. 9A-C) defines a surface area for the fluid to be absorbed by the surrounding tissue, more specifically by the conjunctival tissue on the superior side of the plate body.

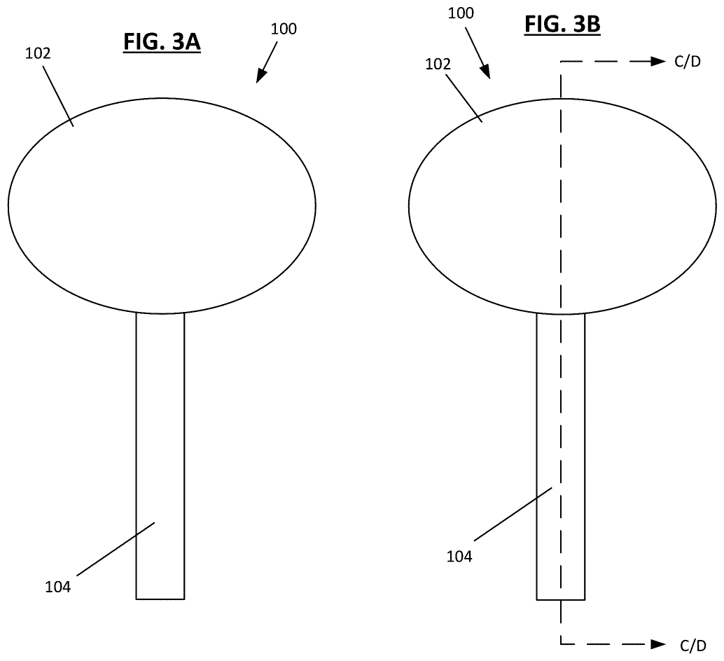

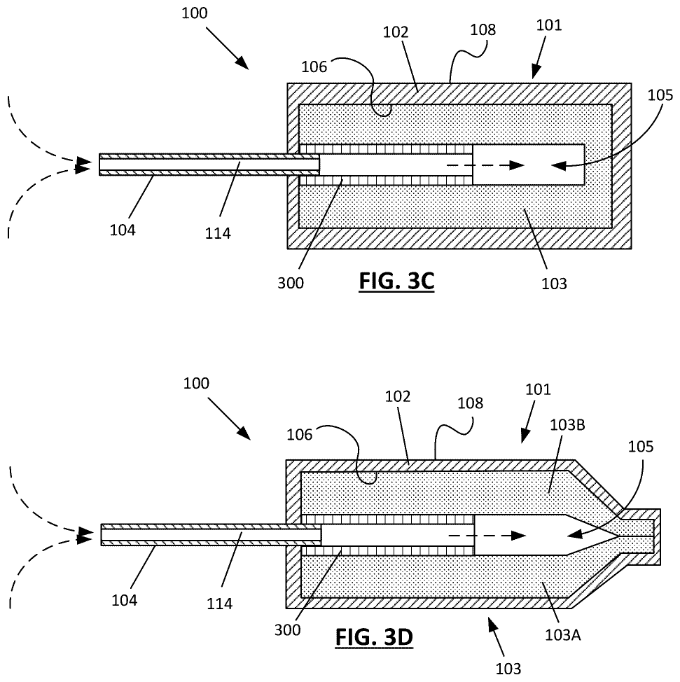

In the Ahmed® device of FIGS. 9A-C, the solid porous polyethylene shell formed using the two layers L1 and L2 of porous material provides an attempt for the shell body to integrate with the surrounding tissue. However, in the process of facilitating integration, the porous shell also causes the problem of allowing the surrounding tissue to infiltrate the inner portion (e.g., the reservoir R), causing the inside of the shell to be covered by tissues, leading to reducing the effect surface area of the bleb for filtration to occur and to possible device failure by clogging up the reservoir R and/or the tube T. Such problems are further explained in the article: Sluch I, et al. “Clinical Experience with the M4 Ahmed Glaucoma Drainage Implant.” J Curr Glaucoma Pract. 2017 September-December; 11(3):92-96. DOI: 10.5005/jp-journals-10028-1231. Epub 2017 Oct. 27. PMID: 29151683; PMCID: PMC5684239. For example, FIG. 3 of the article shows a declining survival probability over time for the M4 model, where survival denotes a patient continuing in the study without experiencing surgical failure. As such, there is a need to overcome the problems addressed above with respect to the devices shown in FIGS. 7A-C, 8A-C, and 9A-C.

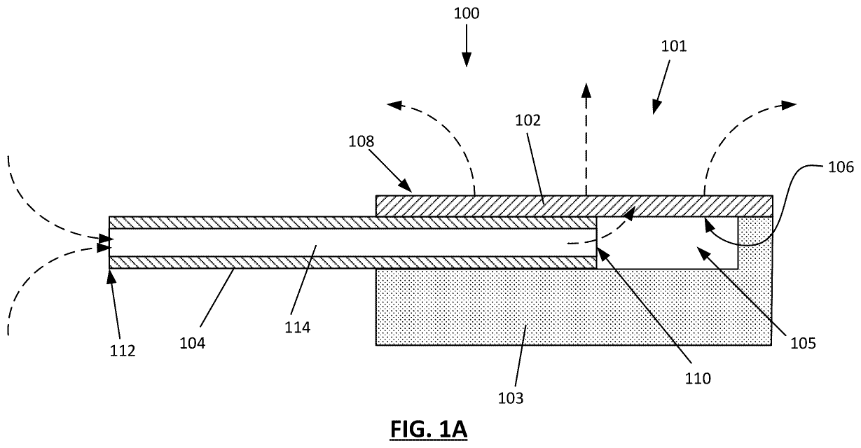

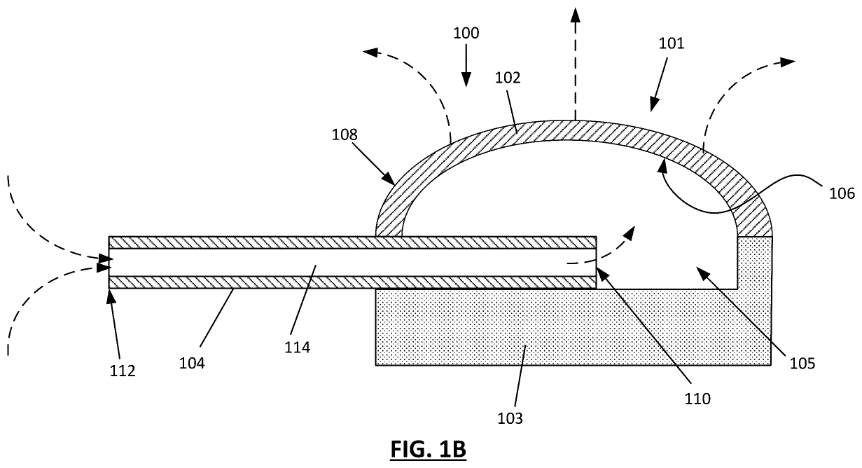

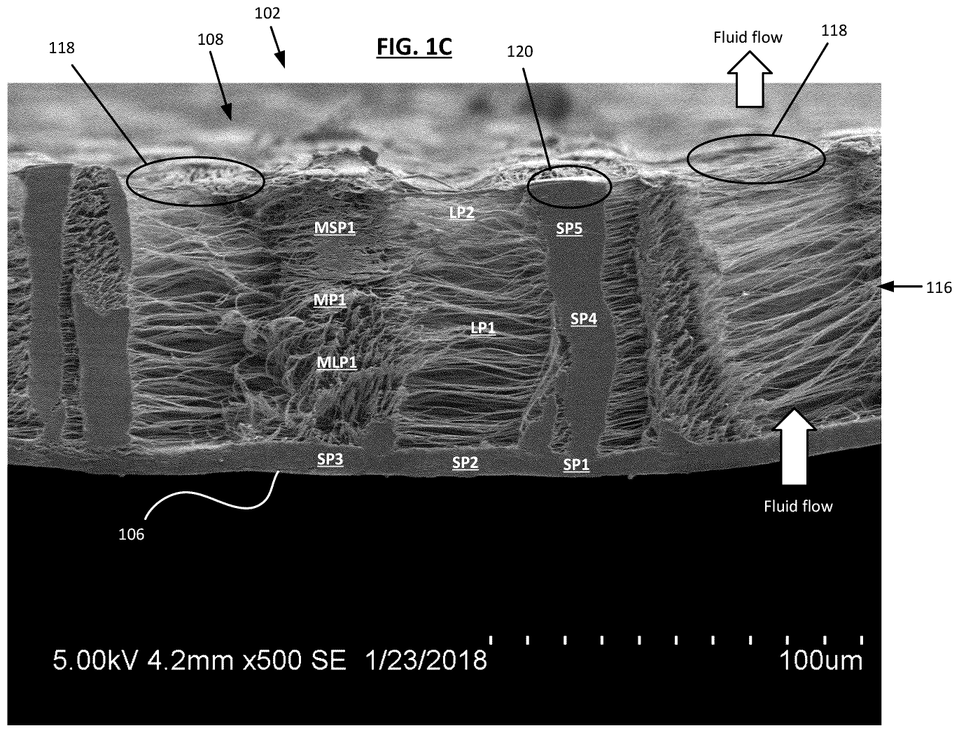

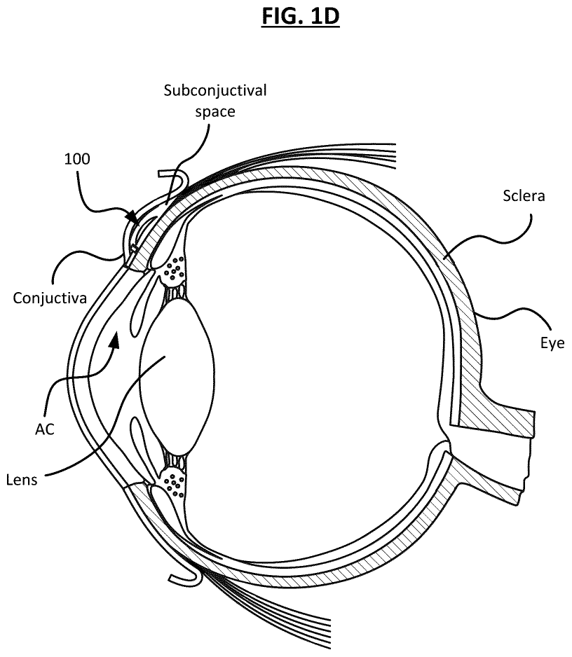

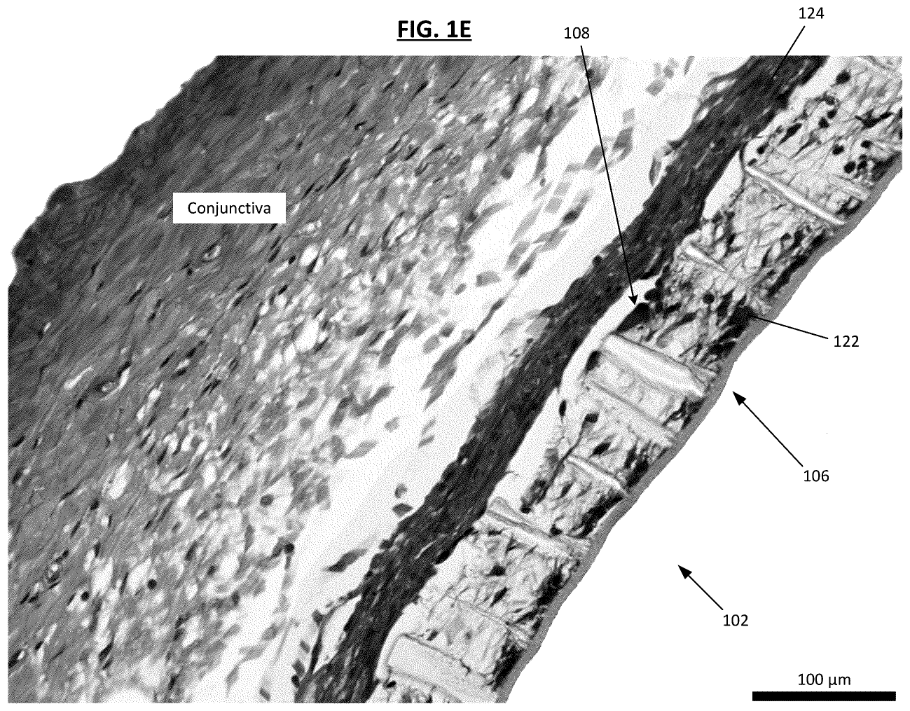

Different embodiments and examples of drainage device 100 for treating glaucoma are disclosed herein. For example, FIG. 1A shows an example of the device 100. As shown, the device 100 is an implantable device which, when implanted at least in part within the tissue of an eye, can be used for draining biological fluid from the eye. In some examples, the fluid may be drained to a tissue surrounding the eye or to a tissue external to the eye. The device 100 includes a body portion 101 and a fluid conduit 104. The body portion 101 is formed using a first body component 102 and a second body component 103, and the first body component 102 has a first surface 106 with a first porosity as well as a second surface 108 with a second porosity that is greater than the first porosity. The second surface 108 opposes (or is disposed in a location of the first body component 102 that is opposite from the location of) the first surface 106. The second body component 103 may have a third porosity that is less than both the first and second porosities of the first body component 102. In some examples, the second body component 103 may be nonporous. In some examples, the first body component 102 may be referred to as a membrane, and the second body component 103 may be referred to as a plate.

The body portion 101 further includes a reservoir 105 fluidly coupled with a lumen 114 of the conduit 104 such that a first end 110 of the conduit 104 is disposed in the reservoir 105 and a second end 112 of the conduit 104 is disposed external to the reservoir 105. In some examples, in forming the reservoir 105, the first body component 102 and the second body component 103 may be heat-treated or sintered to be fused or attached together at the periphery of these body components 102 and 103. In some examples, the heat-treating may be applied to the second body component 103, which may be made using a material such as silicone that has a lower melting point than the material of the first body component 102, in order to fuse the second body component 103 to the first body component 102.

In some examples, thickness of the body portion 101 may range from about 25 μm to about 30 μm, about 30 μm to about 40 μm, about 40 μm to about 50 μm, about 50 μm to about 60 μm, from about 60 μm to about 70 μm, from about 70 μm to about 80 μm, from about 80 μm to about 90 μm, from about 90 μm to about 100 μm, from about 10 μm to about 150 μm, from about 150 μm to about 200 μm, from about 200 μm to about 250 μm, from about 250 μm to about 300 μm, from about 300 μm to about 350 μm, from about 350 μm to about 400 μm, from about 400 μm to about 450 μm, from about 450 μm to about 500 μm, or any other suitable value or range therebetween and/or combination of ranges thereof. In some examples, the body portion 101 may have a diameter in the range of from about 5 mm to about 15 mm, such as about 10 mm for example. In some embodiments, the body portion 101 may be ovular and include a major dimension (e.g., along a major axis of the ellipse) of up to about 30 mm and corresponding minor dimension (e.g., along a minor axis of the ellipse) of up to about 10 mm. As discussed above, given differing anatomies of the human body, the body portion 101 may exceed such dimensions (e.g., 10 mm, 15 mm, and 30 mm) provided that the size does not substantially interfere with normal eye functioning (e.g., pivoting and blinking) or substantially reduce the flexibility of the aqueous humor diffusion member undesirable relative movement occurs between the drainage device 100 and the surrounding tissue when implanted, resulting with a likely consequence of tissue irritation, foreign body tissue response, and/or excessive scar formation. Likewise, the body portion 101 may have a diameter of less than about 5 mm, or even less than 3 mm provided that the body portion 101 is operable to accommodate a sufficient degree of evacuated aqueous humor and is operable to facilitate the reabsorption of aqueous humor to constitute an effective treatment for the patient.

In some examples, the first body component 102 is a membrane with a thickness from about 25 μm to about 50 μm, from about 50 μm to about 75 μm, from about 75 μm to about 100 μm, from about 100 μm to about 125 μm, or any other suitable value or range therebetween or any suitable combination of ranges thereof. The second body component 103 is a plate with a thickness from about 0.8 mm to about 1.0 mm, from about 1.8 mm to about 1.2 mm, from about 1.2 mm to about 1.4 mm, from about 1.4 mm to about 1.6 mm, from about 1.6 mm to about 1.8 mm, from about 1.8 mm to about 2.0 mm, or any other suitable value or range therebetween or any suitable combination of ranges thereof.

In some examples, the first body component 102 is a membrane with a sufficient porosity to allow the fluid from the eye to pass through the membrane and to also reduce or inhibit ingrowth of the tissue external to the eye. The first body component 102 may be more flexible than the second body component 103 which is a plate, such that the body components 102 and 103 have different thicknesses as explained above.

The conduit 104 may be a tube or any suitable construct that allows passage of fluid therethrough. The second end 112 of the conduit 104 is insertable into the eye of the patient to facilitate a drainage of the biological fluid into the conduit 104. Throughout the figures disclosed herein, an arrow with a broken line attached thereto represents the general directions in which fluid may flow with respect to the components of the device 100. For example, in FIG. 1A, fluid may flow into the lumen 114 of the conduit 104 via the second end 112, and after passing through the lumen 114, exit via the first end 110. Thereafter, the fluid may fill the reservoir 105, which may cause the reservoir 105 to inflate as shown in FIG. 1B, and thereafter travel through the material of the first body component 102, and leave the body portion 101 from the second surface 108 into the surrounding environment. In some examples, the first body component 102 is more flexible than the second body component 103 which in some examples may be rigid or stiff, although semi-rigid or flexible second body components are contemplated as well. As such, in some examples, as the reservoir 105 inflates with fluid, only the first body component 102 changes in shape, transitioning from an empty state (FIG. 1A) to a filled state (FIG. 1B) as shown. In some examples, the first body component 102 and the second body component 103 both have sufficient flexibility such that the body portion 101 may be collapsible. The second surface 108 with the second porosity also includes at least one ingrowth surface region 118 which facilitates ingrowth of the tissue as illustrated in FIG. 1C.

FIG. 1C shows a microscopic view of a microporous material of the external surface (second surface 108) of the first body component 102 according to some embodiments. The first body component 102 includes a plurality of internal regions 116 between the first surface 106 and the second surface 108, with porosities that are greater than the first porosity and less than the second porosity. Displayed at the bottom of FIG. 1C is: “5.00 kV 4.2 mm×500 SE Jan. 23, 2018,” and the distance between two consecutive lines as shown at the bottom right hand corner represents 10 μm. For example, the microporous material of FIG. 1C may be referred to throughout with reference to a medical implant device or system. As can be appreciated by a person of skill in the art and with reference to FIG. 1C, the microporous aspects and parameters of the microporous material can be defined in a variety of ways. In an application of a microporous material in an ocular device, such as the device 100 described herein, configured for in situ placement in the tissue of the eye to facilitate the drainage of a biological fluid from the eye (as shown by the white arrows labeled “fluid flow” representing the flow direction of the biological fluid with respect to the first surface 106 and the second surface 108), the microporous properties of such a microporous material can be generally characterized by a volumetric porosity value that can be defined as a ratio of a volume of the air or fluid defined by and contained within the microporous material as compared to an overall volume (or total volume) of the microporous material.

In another definition, a volumetric porosity can be defined as a percentage of the microporous material volume that is occupied by non-structural or transient elements such as air or other fluids. For example, a microporous material with an overall volume of 100 mm³ and with 30 mm³ of that volume comprising chambers holding air or a fluid would have a volumetric porosity value of 0.3 because 30% of the volume of the microporous material is empty or transient space that is filled with air or other fluids.

As can be appreciated, two microporous materials can have the same volumetric porosity but differ in the pore sizes presented to the incoming or exiting air or fluid. For example, a first material can a have a small number of large pores distributed over a fixed overall volume and a second material can have a relatively large number of relatively smaller pores distributed over the same fixed volume, and both microporous materials could have the same volumetric porosity if the air/fluid volume of the two materials are the same.

As can be further appreciated, the properties of the microporous materials used in an ocular drainage device can also be defined by the size of the passages passing through the microporous material or similarly defined as a pore size measured where a passage terminates at a surface of the microporous material or measured along a length of a passage within the material. Microporous materials with small pores or passages can impede flow through the material and comparatively large pores or passages can provide an increased pass through of the air or fluid into, out of, or within the microporous material.

As can be still further appreciated, the properties of the microporous material can also be defined by a tortuosity of the passages entering into and passing through the material, with relatively small or large passages presenting impeded fluid pathways due the frequency of turns in the passages or by the placement of obstructions in the fluid pathways. The air/fluid passthrough rates of a microporous material can be managed by controlling or defining any of the above-described characteristics of the material to provide a suitable material for use to facilitate the drainage of a biological fluid from the eye.

For simplicity, the aforementioned characteristics and variables of the microporous material used in the various embodiments and examples described herein can be presented simply as a porosity which can be based on a volumetric porosity, a pore or passage size, or a tortuosity metric. Again, with reference to FIG. 1C, internal portions of the microporous material can have varying porosities (or volumetric porosities, or pore sizes, or tortuosities). The internal portions can extend between an inner surface (first surface 106) and the external surface (second surface 108).

At any of these portions of the first body component 102, the porosity can comparatively range in degree from small pore size (SP), medium-small pore size (MSP), medium pore size (MP), medium-large pore size (MLP), and large pore size (LP), where LP is larger than MLP, MLP is larger than MP, MP is larger than MSP, and MSP is larger than SP. In some examples, the size of SP may range from about 0.01% to 2%, MSP may range from about 2% to 5%, MP may range from about 5% to 20%, and MLP may range from about 20% to 80% that of the size of LP. The size of SP may range from about 0.01 μm to about 1 μm in pore diameter (or pore average dimension). In some examples, the porosity may increase by about 5 to 10 times as the pore size increases from one category to the next category (for example, from SP to MSP or from MSP to MP, etc.). Assuming, for discussion purposes here, that delivery travels along a relatively straight path through a microporous material so as to sequentially engage porosities of the inner surface 106, a uniform internal portion, and the external surface 108, the combined flow resistance can be represented by likewise concatenating their respective porosities. For instance, the inner surface 106 typically has a low porosity throughout (e.g., to resist tissue ingrowth through the first body component 102, and/or to maintain a plane between the device 100 and the sclera), and portions of the interior portions and the external surface 108 can have any of the aforementioned degrees of porosity. Under these circumstances when the internal portion has a medium porosity and, for example, the internal portions have a medium porosity and the external surface 108 has a high porosity, the drainage of a biological fluid from the eye through the microporous material to tissue surrounding the device can be represented as SP-MP-LP. More examples are discussed here below.

Various delivery paths can be present within the microporous material. Relatively linear flow paths may comprise regions SP1-SP4-SP5, for example or SP3-MLP1-MP1-MSP1. Although some flow paths may be relatively straight, there are also flow paths that are nonlinear. For instance, under certain conditions, at least some flow may proceed to flow through areas of increasingly less resistance such as SP1-LP1-LP2 or SP3-MLP1-LP1-LP2. As will be appreciated, the microstructure of the microporous materials may undergo modification processes to obtain certain types of flow through the microstructure. For instance, the microstructure may have relatively uniform layers across layered within the microstructure, or as shown here, have variable portions throughout the thickness of the microporous material.

In some examples, the first body component 102 defines a wall portion thickness extending between the inner surface 106 and the external surface 108. The wall portion thickness can define an internal region of the first body component 102 having a transition porosity that is between a porosity of the low porosity surface (e.g., having smaller pore sizes) of the inner surface 106 and a porosity of the high porosity surface (e.g., having larger pore sizes) of the external surface 108. In addition, or in alternative, the internal region can have an internal region porosity that is equal to porosities of the low porosity surfaces of the inner surface 106 and the external surface 108. In addition, or in alternative, the internal region can have an internal region porosity that is equal to a porosity of the low porosity surface of the inner surface 106. In addition, or in alternative, the internal region can have an internal region porosity that is equal to a porosity of the high porosity surface of the external surface 108.

With reference still to the microporous material shown in FIG. 1C, the fluid pathways may also be impacted by a pressure difference between the fluid in one area (e.g., at the first surface 106) and the fluid in another area (e.g., at the second surface 108). In some examples, the first surface 106 has the first porosity that prevents or inhibits the ingrowth of the tissue. In some examples, the second surface 108 includes at least one low-porosity region 120 for inhibiting the ingrowth of the tissue. The regions capable of inhibiting tissue ingrowth may have relatively small pore sizes, such as SP and/or MSP as labeled, for example.

Various aspects of the present disclosure relate to drainage devices and methods for biological fluids. For example, the present disclosure addresses devices and methods for draining aqueous humor from an anterior chamber (AC) of an eye (FIG. 1D) of a patient so that the aqueous humor may be resorbed by the body elsewhere. FIG. 1D is an illustration of an eye with a subconjunctival space between a conjunctiva and a sclera of the eye. Implanted within the eye is a drainage system with a drainage device 100 in accordance with principles of the present disclosure. In an aspect of the present disclosure, a mechanism is provided for reabsorption of aqueous humor that has been expelled from the AC of the eye to reduce or otherwise stabilize intraocular pressure. One skilled in the art, however, will appreciate that aspects of the present disclosure are useful in other applications where drainage of biological fluid to be redirected in the body is desired. In some examples, the target implantation locations may include subconjunctival and/or sub-Tenon locations of the eye. In some examples, the device 100 may be at least partially subconjunctivally implanted and at least partially suprachoroidally implanted (e.g., posterior of the pars plana of the eye), as suitable for the intended treatment.

As discussed above, in various embodiments, the fluid conduit is a soft and compliant tubular member insertable into the anterior chamber of the eye. In various example surgical approaches, one or more of the fluid conduit and the aqueous humor diffusion member will be advanced or pushed during the implantation procedure. Soft, thin, and compliant tubular structures are generally difficult to advance through tissue. Accordingly, in various embodiments, the glaucoma drainage devices discussed herein may further include an inner component which may function as a support structure that is integrated with the devices.

In some examples, the device 100 may be implanted ab interno (e.g., from inside the eye), such as through a clear corneal incision, and placed through the sclera and into a dissected subconjunctival space, as those of skill in the art will appreciate. In some other embodiments, the device 100 may be implantable ab externo (e.g., from outside of the eye), such as through a conjunctival incision, as those of skill in the art should appreciate. In some embodiments, a conjunctival radial incision may be performed (e.g., typically near the limbal junction), and blunt dissection of the conjunctiva may performed to expose the sclera and provide a site for placement of aqueous humor diffusion member. In some embodiments, this may require suturing of the aqueous humor diffusion member to the sclera. In some embodiments, a small needle (e.g., typically a 22-gauge or 23-gauge needle), may also be inserted near the scleral spur to provide a track for subsequent insertion and placement of the fluid conduit into the AC. Similarly, it will be appreciated that the means of fluid conduit implantation discussed above may be performed through one or more of an ab interno clear-corneal approach and an ab externo approach.

FIG. 1E is an exemplary image of a histopathology tissue slide showing the first body component 102 and how the conjunctiva tissue of the eye interacts or engages with the first body component 102. A colored image version of FIG. 1E is provided in U.S. Provisional App. No. 63/444,089. In the image, the slide was stained with Masson's Trichrome (a histological stain agent) at 20× magnification (the bar at the lower right-hand corner indicates the scale showing 100 μm). For example, collagen and tissue cells are stained to show different levels of brightness to be visually distinguishable from each other. For example, in FIG. 1E, collagen is shown in a lighter shade of gray (see, for example, the region in the conjunctiva) than the tissue cells, which are shown in a darker shade of gray (see, for example, the tissue cells 122 near the first surface 106 as marked). As shown in FIG. 1E, a layer or band 124 located between the first body component 102 and the conjunctiva includes both the tissue cells and the collagen in a higher density or concentration than in the conjunctiva. It is to be understood that the conjunctiva also includes the tissue cells but in a lower concentration than near the first surface 106. The first body component 102 includes the first surface 106 and the second surface 108, and the second surface 108 includes a plurality of ingrowth surface regions 118 and low-porosity regions 120. The ingrowth surface regions 118 allow tissue cells 122 from the conjunctiva to be received therein in order to facilitate ingrowth of the tissue cells 122 into a portion of the second surface 108, while the low-porosity regions 120 prevent or otherwise substantially inhibit such ingrowth therein. The first surface 106 is free or has an absence of any tissue cell or collagen, or is substantially free thereof, thereby indicating that although the second surface 108 may at least partially facilitate ingrowth of the tissue cells and collagen, the first surface 106 prevents or inhibits the ingrowth of the tissue cells 122 from external to the eye.

FIGS. 2A and 2B show bottom view and top view, respectively, of an example of the drainage device 100, and FIGS. 2C and 2D show two examples or embodiments of the drainage device 100 as seen from the side in a cross-sectional view when cut along the broken line C/D-C/D (both C-C for FIG. 2C and D-D for FIG. 2D).

In the drainage device 100 of FIG. 2C, adhesive 200 (or thermoplastic in some examples) is disposed between the first body component 102 and the second body component 103 to attached the two components together at the periphery to form the reservoir 105 of the body portion 101. The adhesive 200 is also disposed between the first body component 102 and the conduit 104. The adhesive 200 may include any suitable adhesive polymeric material including but not limited to silicone. In some examples, the adhesive 200 is a portion of the second body component 103 that is heat-treated to fuse the portion of the second body component 103 to the first body component 102. The conduit 104 may be attached to the body portion 101 across a portion of a length of a cross-sectional length of the body portion, such as between 10% and 70% of a cross-sectional length of the body portion 101.

In the drainage device 100 of FIG. 2D, an open-ended valve 300 is fluidly coupled with the reservoir 105 such that the valve 300 controls the flow of fluid through the lumen 114. An inner component 302 is also disposed in the reservoir 105 between the first body component 102 and the second body component 103. The inner component 302 is a support structure that is attached to a portion of the first body component 102 and the second body component 103. In some examples, the inner component 302 may be a solid material, a solid and microporous material, a solid material with a microporous membrane attached thereto, or made of multiple components that are not directly attached to each other, as suitable, in order to control inflation of the device 100, or more specifically the inflation of the reservoir 105. In some examples, the inner component 302 may be an adhesive similar to the adhesive 200.

FIGS. 3A and 3B show bottom view and top view, respectively, of an example of the drainage device 100, and FIGS. 3C and 3D show two examples or embodiments of the drainage device 100 as seen from the side in a cross-sectional view when cut along the broken line C/D-C/D (both C-C for FIG. 3C and D-D for FIG. 3D).

In the drainage device 100 of FIG. 3C, the first body component 102 is disposed on the outer surface of the second body component 103 in such a way that the first body component 102 substantially surrounds or envelopes the second body component 103. In some examples, the valve 300 at least partially defines the reservoir 105 and is disposed between the reservoir 105 and the conduit 104 to control the flow of fluid from the lumen 114. Because the first body component 102 is not directly in contact with the fluid within the reservoir 105, the fluid is not capable of passing through the first body component 102 and into the surrounding environment.

In the drainage device 100 of FIG. 3D, the second body component 103 includes two subcomponents 103A and 103B which may be fused or attached together (for example, fusing together via heat-treating or sintering, or attaching with an adhesive or thermoplastic, as suitable) at the periphery to form the second body component 103. As shown in FIG. 3D, at least a portion of the periphery of the subcomponents 103A and 103B may be pinched or pressed together to form the fused periphery for the body portion 101, for example. In both of the examples shown in FIGS. 3C and 3D, the second body component 103 defines the reservoir 105.

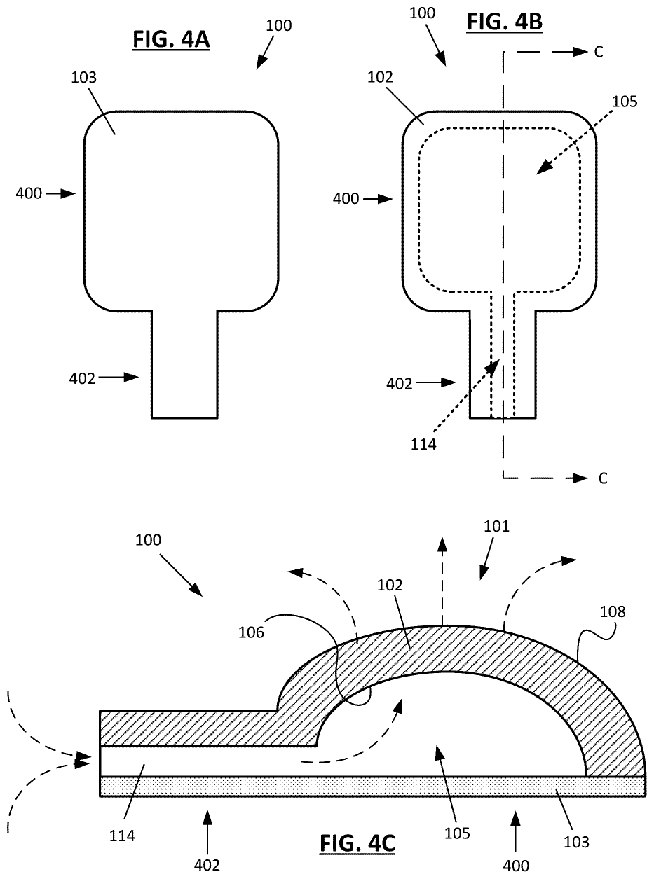

FIGS. 4A and 4B show bottom view and top view, respectively, of an example of the drainage device 100, and FIG. 4C shows an example or embodiment of the drainage device 100 as seen from the side in a cross-sectional view when cut along the broken line C-C. The drainage device 100 in these figures has a body portion 101 which includes two sections: a plate body 400 and a neck or wick section 402. The plate body 400 has a substantially wider width and length than the neck 402. In some examples, the plate body 400 resembles a rectangular shape, although any other suitable shape may be implemented. The first body component 102 and the second body component 103 may be attached or fused at the periphery of both the plate body 400 and the neck 402, such that the plate body 400 defines the reservoir 105 and the neck 402 defines the lumen 114. As such, there is no separate conduit or tube that is inserted between the first body component 102 and the second body component 103, since both the reservoir 105 and the lumen 114 are defined by the first and second body components 102 and 103.

In some examples, the second body component 103 of FIGS. 4A-C is a VisiPlate® implant device developed by Avisi Technologies Inc. (Philadelphia, PA) for treating glaucoma. As such, the second body component 103 may have a substantially smaller thickness than the first body component 102. For example, the first body component 102 may be a membrane with a thickness from about 25 μm to about 50 μm, from about 50 μm to about 75 μm, from about 75 μm to about 100 μm, from about 100 μm to about 125 μm, or any other suitable value or range therebetween or any suitable combination of ranges thereof. The second body component 103 may be a plate with a thickness from about 0.05 μm to about 0.50 μm, from about 0.50 μm to about 1.0 μm, from about 1.0 μm to about 2.0 μm, from about 2.0 μm to about 5.0 μm, from about 5.0 μm to about 8.0 μm, from about 8.0 μm to about 10.0 μm, or any other suitable value or range therebetween or any suitable combination of ranges thereof.

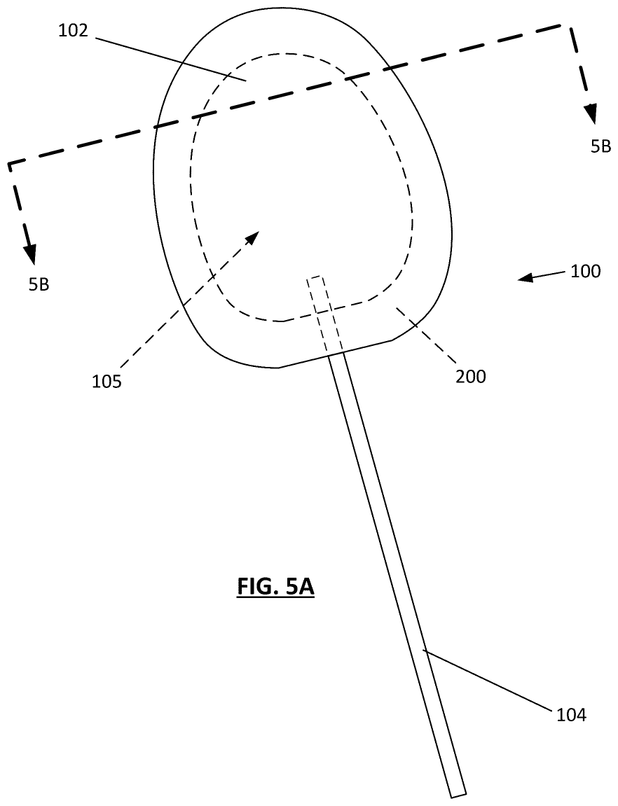

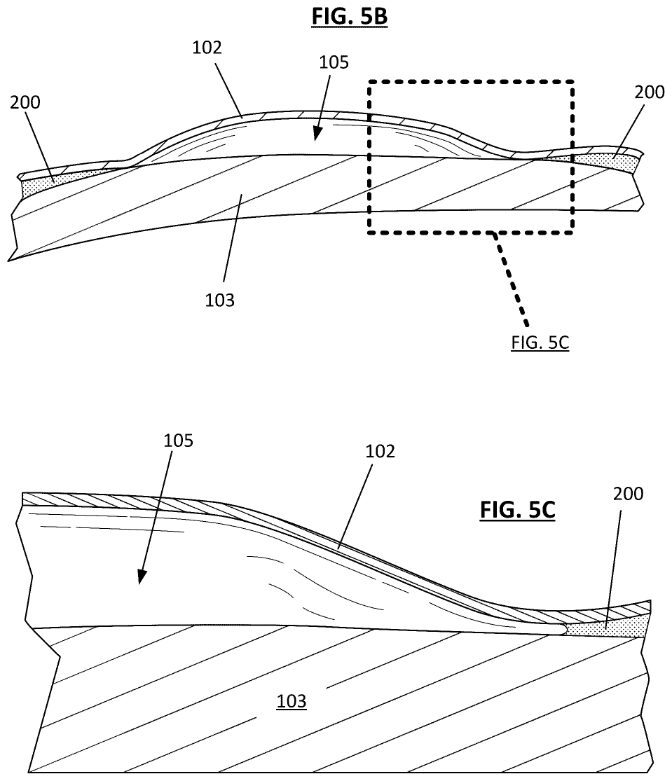

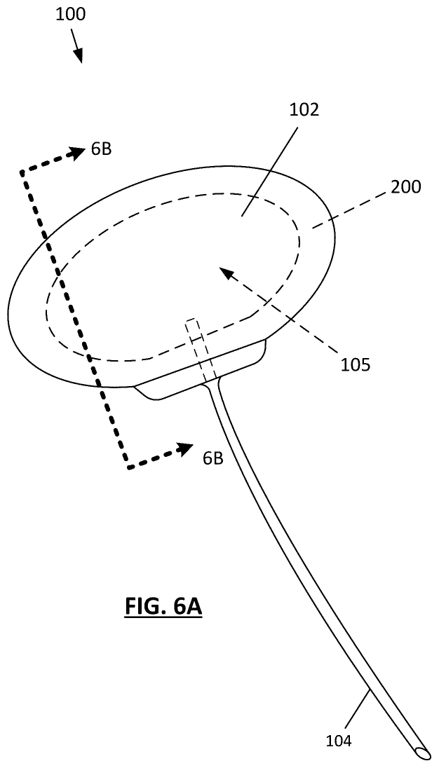

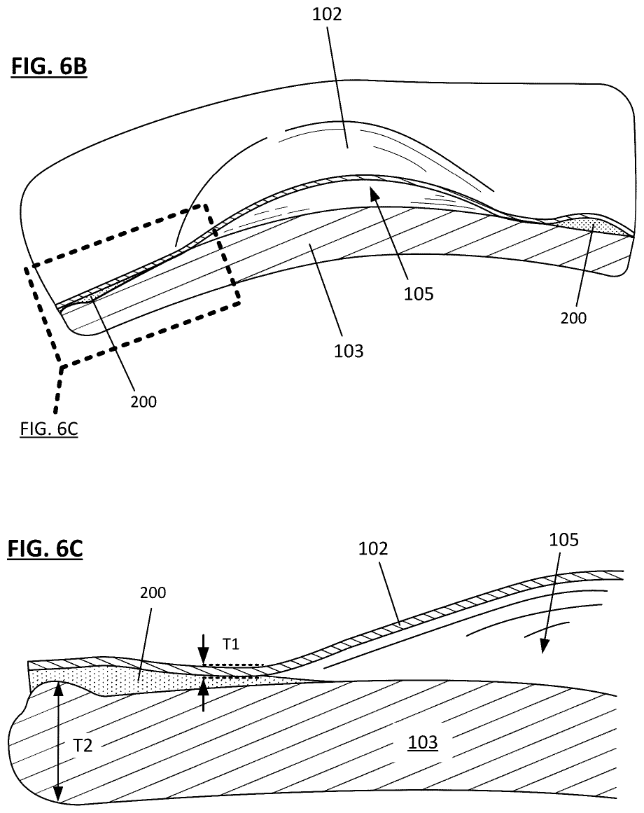

FIGS. 5A-C show an example of the device 100 formed by attaching the first body component 102 to a periphery of the second body component 103, where the second body component 103 is the Ahmed® FP7 device shown in FIGS. 7A-C. FIGS. 6A-C show an example of the device 100 formed by attaching the first body component 102 to a periphery of the second body component 103, where the second body component 103 is the Baerveldt® BG 101-350 device shown in FIG. 8C. The thickness of the first body component 102 (“T1”) and the thickness of the second body component 103 (“T2”) are shown, with adhesive 200 disposed between the two components 102, 103 at the periphery thereof.

In both of the aforementioned examples, the reservoir 105 is formed between the first body component 102 and the second body component 103, such that the first surface 106 of the first body component 102 inhibits the ingrowth of the tissue external to the eye, while the second surface 108 of the first body component 102 may include at least one ingrowth surface region (e.g., the ingrowth surface region 118 as shown in FIG. 1C) for facilitating ingrowth of tissue external to the eye. The periphery or shape of the reservoir 105 may be at least partially defined by the positioning of the adhesive 200 disposed at the periphery between the two components 102 and 103. In some examples, the second surface 108 may also include at least one low-porosity region (e.g., the low-porosity region 120 as shown in FIG. 1C) for inhibiting the ingrowth of the tissue external to the eye.

For example, the body portion 101 (e.g., the body component 102 of the body portion 101, and in some examples, also the body component 103) may include biocompatible materials such as expanded polytetrafluoroethylene (ePTFE). Additionally, the body component(s) may be formed of other biocompatible materials including biocompatible polymers, which may or may not be microporous, including, but not limited to, polyurethane, silicone, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers, expanded polyethylene (ePE), and polytetrafluoroethylene (PTFE).

At least the first body component 102 (and in some examples also the second body component 103) may be in the form of one or more sheets or films, and the sheets or films may include knitted, woven, and/or non-woven forms including individual or multi-fiber strands. In some embodiments, at least the first body component 102 may be formed from a plurality of sheets or films of polymer material. In some embodiments, the sheets or films may be laminated or otherwise mechanically coupled together to form the first body component 102 (and in some examples also the second body component 103) of the body portion 101. Coupling of the sheets or films may be accomplished by a variety of mechanisms, including heat treatment, high pressure compression, bonding agents such as one or more adhesives, lamination, or other suitable methods known to one of skill in the art.

In some embodiments, the body components 102 and 103 may be partially or completely bonded via thermal methods (e.g., where one or both of the polymers forming the materials are brought to or above their melting temperature). In some embodiments, such thermal processes facilitate adhesive or cohesive bond formation between the materials or layers of material. In some embodiments, the body components 102 and 103 and/or the layers of material forming the first body component 102 may be partially bonded via thermal methods where at least one of the materials is brought to or above its melting temperature. Such thermal processes may facilitate adhesive or cohesive bond formation between the materials or layers of material. In some embodiments, one or more suitable adhesives are utilized and provide a sufficiently bonded interface. The body components 102 and 103 may be coupled together at one or more discrete locations (such as the periphery) to form stabilizing structures that extend through the resulting structure.

In some examples, the tube or conduit 104, the valve 300, and/or the support structure such as the inner component 302 may be formed of material or materials including but not limited to: PTFE, ePTFE, urethanes, polyurethane, silicones (organopolysiloxanes), polysulfone, PVDF, PHFP, PFA, polyolefin, FEP, ethylene fluorinated ethylene-propylene (EFEP), ethylene-tetrafluoroethylene (ETFE), and acrylic copolymers, among others. In some embodiments, the materials may include other biocompatible polymers suitable for use in forming the conduit 104, the valve 300, and/or the inner component 302 including, but not limited to, copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of any of the foregoing may be used. In various embodiments, the elastomer or elastomeric material may include perfluoromethyl vinyl ether and tetrafluoroethylene, (per)fluoroalkylvinylethers (PAVE), a copolymer of tetrafluoroethylene and perfluoromethyl vinyl ether, silicone, a fluoroelastomer, a urethane, butyl rubber, styrene-butadiene, isobutylene-isoprene, or a tetrafluoroethylene (TFE)/perfluoromethyl vinyl ether (PMVE) copolymer. In some examples, the valve 300 and/or the inner component 302 may be formed using thermoplastics including, but are not limited to, acrylic and its copolymers, polyester and its copolymers, polypropylene, polystyrene, nylon and its copolymers, PTFE, among others.

Advantages of implementing the embodiments of the drainage device as disclosed herein include, but are not limited to, overcoming the problems as addressed above with respect to the prior-art glaucoma treatment devices shown in FIGS. 7A-C, 8A-C, and 9A-C, by providing a sufficient means of tissue integration for the plate body to reduce or eliminate the risk of fibrosis, while also reducing or preventing the surrounding tissue from infiltrating the internal reservoir of the device to reduce or eliminate the risk of device failure or malfunction. For example, as shown in FIGS. 5A-C, the Ahmed® FP7 device may be improved upon by forming the reservoir 105 using the first body component 102 such that the first surface 106 of the first body component 102 inhibits the ingrowth of tissue external to the eye. Also, for example, as shown in FIGS. 6A-C, the Baerveldt® BG 101-350 device may similarly be improved upon by forming the reservoir 105 using the first body component 102 to provide similar benefits.

Persons skilled in the art will readily appreciate that various aspects of the present disclosure may be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A drainage device for draining a fluid from an eye to a tissue external to the eye, the drainage device being implantable at least in part within a tissue of the eye and comprising:
a body portion comprising:
a membrane having a first surface with a first porosity and a second surface with a second porosity that is greater than the first porosity, the second surface opposing the first surface, and
a plate having a porosity less than the first porosity,
wherein the membrane is more flexible than the plate,
wherein the membrane has a first thickness from about 25 μm to about 125 μm, and the plate has a second thickness from about 0.8 mm to about 2.0 mm,
the membrane and the plate defining a reservoir of the body portion; and
a conduit having an end portion fluidly coupled with the reservoir and insertable into the eye to facilitate a drainage of the fluid into the conduit,
wherein the second surface of the membrane with the second porosity includes at least one ingrowth surface region for facilitating ingrowth of tissue external to the eye.

2. The drainage device of claim 1, wherein the plate is nonporous.

3. The drainage device of claim 1, the plate comprising one or more of: silicone or polypropylene.

4. The drainage device of claim 1, the membrane comprising one or more of: expanded polytetrafluoroethylene (ePTFE), expanded polyethylene (ePE), silicone, polysulfone, polyvinylidene fluorine (PVDF), polyhexafluoropropylene (PHFP), perfluoroalkoxy polymer (PFA), polyolefin, fluorinated ethylene propylene (FEP), or acrylic copolymer.

5. The drainage device of claim 1, further comprising an adhesive disposed between the membrane and the plate.

6. The drainage device of claim 5, wherein the adhesive is a thermoplastic.

7. A drainage device for draining a fluid from an eye to a tissue external to the eye, the drainage device being implantable at least in part within a tissue of the eye and comprising:
a body portion comprising:
a membrane having a porosity such that the membrane is configured to allow the fluid from the eye to pass therethrough and inhibit ingrowth of the tissue external to the eye, and
a plate having a porosity less than the porosity of the membrane,
wherein the membrane is more flexible than the plate,
wherein the membrane has a first thickness from about 25 μm to about 125 μm, and the plate has a second thickness from about 0.8 mm to about 2.0 mm,
the membrane and the plate defining a reservoir of the body portion; and
a conduit having an end portion fluidly coupled with the reservoir and insertable into the eye to facilitate a drainage of the fluid into the conduit.

8. The drainage device of claim 7, wherein the plate is nonporous.

9. The drainage device of claim 7, the plate comprising one or more of: silicone or polypropylene.

10. The drainage device of claim 7, the membrane comprising one or more of: expanded polytetrafluoroethylene (ePTFE), expanded polyethylene (ePE), silicone, polysulfone, polyvinylidene fluorine (PVDF), polyhexafluoropropylene (PHFP), perfluoroalkoxy polymer (PFA), polyolefin, fluorinated ethylene propylene (FEP), or acrylic copolymer.

11. The drainage device of claim 7, further comprising an adhesive disposed between the membrane and the plate.

12. The drainage device of claim 11, wherein the adhesive is a thermoplastic.

13. A drainage device for draining a fluid from an eye to a tissue external to the eye, the drainage device being implantable at least in part within a tissue of the eye and comprising:
a body portion comprising:
a membrane having a first surface with a first porosity and a second surface with a second porosity that is greater than the first porosity, the second surface opposing the first surface, and
a plate having a porosity less than the first porosity,
wherein the membrane has a first thickness from about 25 μm to about 125 μm, and the plate has a second thickness from about 0.05 μm to about 10 μm,
the membrane and the plate defining a reservoir of the body portion; and
a conduit at least partially defined by the membrane and the plate, the conduit fluidly coupled with the reservoir and insertable into the eye to facilitate a drainage of the fluid into the conduit,
wherein the second surface of the first body component with the second porosity includes at least one ingrowth surface region for facilitating ingrowth of tissue external to the eye.

14. The drainage device of claim 13, wherein the plate is nonporous.

15. The drainage device of claim 13, the plate comprising one or more of: silicone or polypropylene.

16. The drainage device of claim 13, the membrane comprising one or more of: expanded polytetrafluoroethylene (ePTFE), expanded polyethylene (ePE), silicone, polysulfone, polyvinylidene fluorine (PVDF), polyhexafluoropropylene (PHFP), perfluoroalkoxy polymer (PFA), polyolefin, fluorinated ethylene propylene (FEP), or acrylic copolymer.

17. The drainage device of claim 13, further comprising an adhesive disposed between the membrane and the plate.

18. The drainage device of claim 17, wherein the adhesive is a thermoplastic.

19. A drainage device for draining a fluid from an eye to a tissue external to the eye, the drainage device being implantable at least in part within a tissue of the eye and comprising:
a collapsible body portion comprising:
a first body component having a first surface with a first porosity and a second surface with a second porosity that is greater than the first porosity, the second surface opposing the first surface, and
a second body component having a third porosity that is less than the first porosity,
the first and second body components defining a reservoir of the collapsible body portion; and
a conduit having an end portion fluidly coupled with the reservoir and insertable into the eye to facilitate a drainage of the fluid into the conduit,
wherein the second surface of the first body component with the second porosity includes at least one ingrowth surface region for facilitating ingrowth of tissue external to the eye.

20. The drainage device of claim 19, wherein the second body component is nonporous.

21. The drainage device of claim 19, the second body component comprising one or more of: silicone or polypropylene.

22. The drainage device of claim 19, the second body component having a greater stiffness than first body component.

23. The drainage device of claim 19, the first body component comprising one or more of: expanded polytetrafluoroethylene (ePTFE), expanded polyethylene (ePE), silicone, polysulfone, polyvinylidene fluorine (PVDF), polyhexafluoropropylene (PHFP), perfluoroalkoxy polymer (PFA), polyolefin, fluorinated ethylene propylene (FEP), or acrylic copolymer.

24. The drainage device of claim 19, further comprising an adhesive disposed between first and second body components.

25. The drainage device of claim 24, wherein the adhesive is a thermoplastic.

26. A drainage device for draining a fluid from an eye to a tissue external to the eye, the drainage device being implantable at least in part within a tissue of the eye and comprising:
a body portion comprising:
a first body component having a first surface with a first porosity and a second surface with a second porosity that is greater than the first porosity, the second surface opposing the first surface, and
a second body component having a third porosity that is less than the first porosity and defining a reservoir of the body portion, wherein the first surface of the first body component is attached to an outer surface of the second body component; and
a conduit having an end portion fluidly coupled with the reservoir and insertable into the eye to facilitate a drainage of the fluid into the conduit,
wherein the second surface of the first body component with the second porosity includes at least one ingrowth surface region for facilitating ingrowth of tissue external to the eye, and the first surface of the first body component with the first porosity is configured to inhibit the ingrowth of tissue external to the eye.

27. The drainage device of claim 26, the second body component comprising a plurality of subcomponents attached together.

28. The drainage device of claim 26, further comprising an open-ended valve enclosed in the body portion, fluidly coupled with the conduit, and partially defining the reservoir.

29. The drainage device of claim 26, wherein the first surface with the first porosity is configured to inhibit the ingrowth of the tissue external to the eye.

30. The drainage device of claim 26, wherein the second surface further includes at least one low-porosity region for inhibiting the ingrowth of the tissue external to the eye.

31. The drainage device of claim 26, wherein the first body component comprises a plurality of internal regions with porosities that are greater than the first porosity and less than the second porosity.

32. The drainage device of claim 26, wherein the conduit is attached to a periphery of the body portion.

33. The drainage device of claim 26, wherein the conduit is attached to the body portion across a portion of a cross-sectional length of the body portion.
